(19) United States
(12) Patent Application Publication
(10) Pub. No.: US 2023/0052152 A1
(43) Pub. Date: Feb. 16, 2023

(54) COMPOUNDS FOR TREATMENT OF ALZHEIMER'S DISEASE

(71) Applicants: SULFATEQ B.V., GRONINGEN (NL); RIJKSUNIVERSITEIT GRONINGEN, GRONINGEN (NL)

(72) Inventors: Robert Henk HENNING, GRONINGEN (NL); Adrianus Cornelis VAN DER GRAAF, GRONINGEN (NL); Guido KRENNING, GRONINGEN (NL); Daniël Henri SWART, LEEUWARDEN (NL); Christina Françoise DE VEIJ MESTDAGH, AMSTERDAM (NL); Pieter Cornelis VOGELAAR, GRONINGEN (NL)

(21) Appl. No.: 17/783,671

(22) PCT Filed: Dec. 11, 2020
(86) PCT No.: PCT/NL2020/050782

§ 371 Date: Jun. 9, 2022

(30) Foreign Application Priority Data
Dec. 11, 2019 (NL) 2024431

(51) Int. Cl. A61K 31/496; A61K 31/453; A61K 31/495; A61P 25/28

(52) U.S. Cl. CPC A61K 31/496; A61K 31/453; A61K 31/495; A61P 25/28

(57) ABSTRACT

The invention relates to certain chromanol, quinone or hydroquinone compounds and derivatives thereof for treatment of Alzheimer's disease and/or for improving memory function and/or reducing plaque load. Specifically, the present invention relates to chromanol compounds chosen from (6-hydroxy-2,5,7,8-tetramethylchroman-2yl)(piperazin-1-5 yl)methanone, ((S)-6-hy-droxy-2,5,7,8-tetramethyl-N-((R)-piperidin-3-yl)chroman-2-carboxamide hydrochloride and S-(6-hydroxy-2,5,7,8-tetramethylchro-man-2-yl)(4-(2-hydroxyethyl)piperazin-1-yl)methanone, and pharmaceutically acceptable salts thereof.

### I. FIELD OF THE INVENTION

The invention relates to compounds for treatment of Alzheimer's disease. The invention further relates to chromanol compounds and derivates thereof for improving the memory function.

### II. DESCRIPTION OF THE BACKGROUND ART

Alzheimer's disease is a progressive neurodegenerative disorder and the leading cause of dementia in the elderly.

EP 2994160 B1 discloses a method for the treatment of Alzheimer's disease in patients having moderate Alzheimer's disease and/or carrying an ApoE4 allele by administration of pooled immunoglobulin G.

EP 2892563 B1 describes methods of treating Alzheimer's disease as adjunctive therapy to acetylcholinesterase treatment comprising administering an effective daily dose of N-(2-(6-fluoro-1H-indol-3-yl)ethyl)-3-(2,2,3,3-tetrafluopropropoxy)benzylamine or a pharmaceutically acceptable salt to a patient in need of such treatment, wherein the effective daily dose administered to the patient is between about 30 and about 60 mg.

EP 2937085 B1 describes that a combination of 6-[4-(1-cyclohexyl-1 H-tetrazol-5 5-yl)butoxy]-3,4-dihydrocarbostyril (cilostazol) or a salt thereof, and donepezil or a salt thereof exhibits synergistic action for treating Alzheimer's disease.

WO2002/043666 prophetically suggests that the use of antioxidants can prevent or reduce mental deterioration. Although antioxidants indeed may lower the oxidative burden in mitochondria, a clear effect in treating Alzheimer is not found.

Cai et al. in ACS Chemical Neuroscience (2017) 8:2496-2511 describe medicaments based on donepezil substituted with a Trolox moiety, suggested for use in the treatment of Alzheimer. Several in vitro tests suggest some activity for some biomarkers of Alzheimer.

Amyloid beta (Aβ or Abeta) denotes peptides of 36-43 amino acids that are the main component of the amyloid plaques found in the brains of people with Alzheimer's disease. The peptides derive from the amyloid precursor protein (APP), which is cleaved by beta secretase and gamma secretase to yield Aft AP molecules can aggregate to form flexible soluble oligomers which may exist in several forms. It is now believed that certain misfolded oligomers (known as “seeds”) can induce other AP molecules to also take the misfolded oligomeric form, leading to a chain reaction resulting in plaque formation. The soluble oligomers are toxic to nerve cells, and plaques form from soluble oligomers.

There remains a need for new compounds for treatment of Alzheimer's disease and related diseases linked to a deterioration of the mitochondrial function and health, in particular ones that have less side effects, or preferably no side effects at all in the dosing range of such compound.

It is an object of the present invention to provide compounds for the treatment of Alzheimer's disease.

It is a further object of the present invention to provide compounds for improving the memory function.

It is a further object of the present invention to provide compounds for reducing the development of beta-plaque load in a patient that is experiencing Alzheimer disease.

### III. BRIEF SUMMARY OF THE INVENTION

One or more of the above objects are met by providing certain chromanol, quinone or hydroquinone compounds for one or more of said treatments.

The above objects are met by the present invention by providing compounds according to formula (I), (II), the hydroquinone analogue of formula (II), or a pharmaceutically acceptable salt thereof, for use in the treatment of Alzheimer's disease or for improving the memory function, and/or for reducing plaque load in an Alzheimer disease patient;

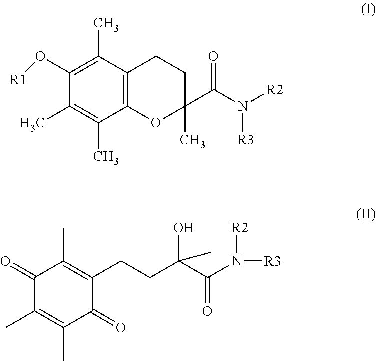

wherein R1 represents a hydrogen or prodrug moiety that can be removed in living tissue
and wherein either
   R2 and R3 together with the N atom to which they are attached form a saturated or unsaturated, non-aromatic, optionally substituted, 5-8 membered ring, having one to four N, O, or S atoms, wherein R2 and R3 together contain 3-12 carbon atoms;
   or R2 is a hydrogen atom, or an alkyl group with 1-6 carbon atoms, and R3 is an alkyl group, optionally substituted with nitrogen or oxygen, wherein the alkyl group comprises 3-12 carbon atoms, the alkyl group in R3 comprises one or more non-aromatic cyclic structures and may contain linear and/or branched groups, and one or more ethylenic unsaturations.

For the present invention, the compound according to formula (II) includes the hydrogenated quinone (i.e. the hydroquinone) analogue, although the quinone derivative is preferred in view of stability.

In a preferred embodiment, the nitrogen can be amine, quaternary amine, guanidine or imine and oxygen is hydroxyl, carbonyl or carboxylic acid; and/or oxygen and nitrogen together may form amide, urea or carbamate groups.

In a preferred embodiment, R1 in formula (I) is hydrogen or forms together with the 6-oxygen an ester group with 2-6 carbon atoms.

In a preferred embodiment of either compounds according to formula (I) or according to formula (II), R2 and R3 together with the N atom to which they are attached form a saturated ring incorporating an additional N atom, which ring is unsubstituted or substituted with an alcohol, or alkanol group having 1-4 carbon atoms, such as ethylol.

In another preferred embodiment, R2 is a hydrogen atom and R3 comprises a saturated cyclic structure having 4-7 carbon atoms and having one nitrogen atom, which ring is unsubstituted or substituted with an alcohol, or alkanol group having 1-4 carbon atoms, such as ethylol.

According to yet another preferred embodiment, the compound is either (6-hydroxy-2,5,7,8-tetramethylchroman-2yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13), or (6-hydroxy-2,5,7,8-tetramethylchroman-2-yl)(4-(2-hydroxyethyl)piperazin-1-yl)methanone (SUL-109).

In a most preferred embodiment, the compound is the S-enantiomer of SUL-109, namely S-(6-hydroxy-2,5,7,8-tetramethylchroman-2-yl)(4-(2-hydroxyethyl)piperazin-1-yl)methanone (SUL-138).

In a preferred embodiment according to the invention, the compound either according to formula (I) or according to formula (II) has a molecular weight lower than 500 Da.

As such, Trolox derivatives are described, like for example in WO2014/098586, WO2014/011047 and WO2017/060432. However, memory function or plaque formation are not investigated, nor another type of in vivo or in vitro test directly relevant for the treatment of Alzheimer disease.

WO2019/101826 suggests that some compounds comprising a Trolox moiety may act as MPGES inhibitor, which is suggested to be of advantage in treating inflammatory diseases. WO2019/101826 suggests that Alzheimer disease may act via MPGES, however, our research has not found any difference in expression in wild type-mice versus APP/PS1 mice, which indicates that MPGES is not relevant for Alzheimer disease.

Memory function and plaque formation caused by polymerization of amyloid-β are considered main issues with Alzheimer disease. Despite the fact that some antioxidants possibly reduce underlying oxidation mechanisms, no evidence has been provided that actually the memory function can be improved. The present findings show that specific Trolox derivatives can be a valuable new treatment option for treating Alzheimer Disease.

### IV. SHORT DESCRIPTION OF THE FIGURES

FIG. 1 shows how chronic SUL-138 treatment increases memory (Freezing %) in WT and APP mice

FIG. 2 shows how SUL-138 increases LTP maintenance in both WT and APP mice.

FIG. 3 shows that SUL-138 treatment reduces plaque numbers and size in APP/PS1 mice.

### V. DETAILED DESCRIPTION OF THE INVENTION

One or more of the above objects are met by the present invention by providing compounds according to formula (I) or (II), as shown above, or a pharmaceutically acceptable salt thereof for use in the treatment of Alzheimer's disease or for improving the memory function and/or for reducing plaque load in a patient experiencing Alzheimer disease.

Preferably, memory function is improved, while also plaque formation is reduced, thereby allowing an even further improved treatment of Alzheimer Disease.

As far as improving the memory function is not considered a medical treatment, the present invention also provides for the use of the compounds as defined for the improvement of the memory function in a mammal. The mammal preferably is a human.

R1 can be a substituent that is easily removed in the human body, such that the compound is a prodrug. R1 can be for example an amino acid derivative or ester derivative, and generally has a molecular weight lower than 100 dalton.

In a preferred embodiment, R1 in formula (I) is hydrogen or forms together with the 6-oxygen an ester group with 2-6 carbon atoms. The ester can comprise one or more ether or alcohol groups. Suitable esters are acetate, butyrate, 3-hydroxy butyrate and the like. In a preferred embodiment of either compounds according to formula (I) or according to formula (II), R2 and R3 together with the N atom to which they are attached form a saturated ring having 3-6 carbon atoms and incorporating one additional N atom, which may be substituted with 1-4 carbon atoms that may comprise an oxygen, carboxylic acid or amine group.

More preferably, R2 and R3 together with the N atom to which they are attached form a 5-7 membered ring comprising one additional amine group, which ring is optionally substituted with methyl, ethyl, or alcohol substituted methyl or ethyl.

In another preferred embodiment, R2 is a hydrogen atom and R3 comprises a cyclic structure having 3-6 carbon atoms and having one nitrogen atom.

More preferably, R2 is a hydrogen atom, and R3 comprises a 5-7 membered ring comprising one additional amine group, which ring is attached to the amide-nitrogen, and which ring is optionally substituted with methyl, ethyl, or alcohol substituted methyl or ethyl.

In either case, the ring (the cyclic structure formed by R2 and R3, or of R3 alone) may be unsubstituted or substituted with an alkyl having 1-4 carbon atoms, alcohol, or alkanol group having 1-4 carbon atoms, such as ethylol.

In a preferred embodiment according to the invention, the compound either according to formula (I) or according to formula (II) has a molecular weight lower than 500 Da.

Certain chromanol compounds have been described in WO2014/098586. The compounds described in detail have abbreviations, referring to SUL-XXX (XXX being a 2 or 3 digit number). Many of these compounds are racemic mixtures, although some enantiomers have been tested as well. Suitable methods to prepare chromanol compounds according to the present invention are described in WO2014/098586 or WO2014/011047.

WO 2017060432 A1 discloses amide-derivatives of 2-hydroxy-2-methyl-4-(3,5,6-trimethyl-1,4-benzoquinon-2-yl)-butanoic acid and methods of making such compounds.

Hydrogenated quinone derivatives can be easily prepared by hydrogenation of the quinone structure.

According to yet another preferred embodiment, the compound is either (6-hydroxy-2,5,7,8-tetramethylchroman-2yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13), or (6-hydroxy-2,5,7,8-tetramethylchroman-2-yl)(4-(2-hydroxyethyl)piperazin-1-yl)methanone (SUL-109).

In a most preferred embodiment, the compound is the S-enantiomer of SUL-109, namely S-(6-hydroxy-2,5,7,8-tetramethylchroman-2-yl)(4-(2-hydroxyethyl)piperazin-1-yl)methanone (SUL-138).

The counterion in the pharmaceutically acceptable salt can be a counterion as known in the art. Preferably, the compounds have at least one basic nitrogen, an amine, which can be protonated. The counterion preferably is a halogen such as chloride, sulphate, citrate, formate or the like, and most preferably chloride.

The compounds are effective as a racemic mixture or in a substantially pure enantiomeric form. The compounds have one or more chiral centers, generally one or two.

Preferably, the compound is a substantially enantiomerically pure compound. Substantially enantiomerically pure is about 95% enantiomeric excess or more, more preferably about 98% enantiomeric excess, and most preferably about 99% or more enantiomeric excess. Also, in case the compound contains more than one chiral center, these amounts apply.

The compounds are preferably used in effective amounts, to achieve an improvement in memory function and/or to achieve treatment of Alzheimer's disease.

The term ‘treatment’ encompasses reduction in progress of the disease and/or improvement in symptoms of the disease.

Effects generally are observed with amounts of about 1 μM in body fluid, but preferably higher amounts are used. Preferred amounts are concentrations in vivo or in vitro of about 10 μM or higher, more preferably about 20 μM or higher. Generally, a concentration in human of about 200 μM or lower should be sufficient and safe.

For human use, this would mean—assuming a 30 L distribution volume, 100% availability and a concentration of about 1 μM—a dosage of about 10 mg or more. Preferred amounts would result in a concentration of about 10 μM—for which a dosage of about 100 mg or more would be suitable. Hence, preferably, dosage forms of about 20 mg or more, preferably 50 mg or more, preferably 100 mg or more are suitable. Generally, solid, oral dosage forms contain as a maximum about 500 mg compound, preferably about 450 mg or less, to allow for excipients. With i.v. other liquid forms of administration, larger amounts can be administered.

Examples of dosages which can be used are an effective amount of the compounds of the invention of a dosage of 0.2 mg/kg or higher, such as preferably within the range of about 1 mg/kg to about 100 mg/kg, or within about 2 mg/kg to about 40 mg/kg body weight, or within about 3 mg/kg to about 30 mg/kg body weight, or within about 4 mg/kg to about 15 mg/kg body weight. Compounds of the present invention may be administered in a single daily dose, or the total daily dosage may be administered in divided dosage of two, three or four times daily.

The compounds described herein can be formulated as pharmaceutical compositions by formulation with additives such as pharmaceutically or physiologically acceptable excipients carriers, and vehicles.

Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidone, low melting waxes, and the like, as well as combinations of any two or more thereof. Other suitable pharmaceutically acceptable excipients are described in “Remington's Pharmaceutical Sciences,” Mack Pub. Co., New Jersey (1991).

A pharmaceutical composition preferably comprises a unit dose formulation, where the unit dose is a dose sufficient to have a therapeutic effect. The unit dose may be a dose administered periodically in a course of treatment or suppression of a disorder.

In addition, the unit dose may be a dose administered periodically in a course of treatment to improve native cognitive functions related to memory.

The compounds of the invention may be administered enterally, orally, parenterally, sublingually, by inhalation (e.g. as mists or sprays), rectally, or topically in dosage unit formulations containing conventional nontoxic pharmaceutically or physiologically acceptable carriers, adjuvants, and vehicles as desired. The term parenteral as used herein includes subcutaneous injections, intravenous, intramuscular, intratarsal injection, or infusion techniques. The compounds are mixed with pharmaceutically acceptable carriers, adjuvants, and vehicles appropriate for the desired route of administration.

Oral administration is a preferred route of administration, and formulations suitable for oral administration are preferred formulations.

The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at room temperature but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavouring, and perfuming agents.

The amount of active ingredient that may be combined with the carrier materials to produce a single dosage form will vary depending upon the host to which the active ingredient is administered and the particular mode of administration. The unit dosage chosen is usually fabricated and administered to provide a defined final concentration of drug in the blood, tissues, organs, or other targeted region of the body. The effective amount for a given situation can be readily determined by routine experimentation and is within the skill and judgment of the ordinary clinician or skilled person.

The present invention will be further illustrated using the examples below. In the examples, reference is made to figures.

### VI. Examples

### Example 1

The effectiveness of the compounds according to the invention for treatment of Alzheimer's disease was tested by two independent tests: one reflecting memory and one showing synaptic connectivity.

### Methods and Experimental Details

The APP/PS1 mouse model is a widely used A-beta pathology model for Alzheimer's disease (AD) (1 of the 2 main neuropathological hallmarks of AD). These mice contain human transgenes for APP (Swedish mutation) and PSEN1 (L166P mutation), which will lead to pathological amyloid deposition in the brain and impairments in hippocampal dependent memory and Long Term Potentiation (LTP) starting at ˜3 months of age (3 moa).

The effectiveness of SUL-138 ((6-hydroxy-2,5,7,8-tetramethylchroman-2-yl)(4-(2-hydroxyethyl)piperazin-1-yl)methanone) in relieving/preventing common pathology in the APP/PS1 model was tested. The effect on memory was tested in a hippocampal dependent context test (Fear conditioning (FC)) and synaptic connectivity was tested via electrophysiological LTP (Long-term potentiation) measurements. Both are impaired in this mouse model under basal conditions. In addition, Phenotypers (Sylics) were used to exclude that SUL-138 induces atypical behavior after chronic oral treatment.

Wild type (WT) and APP/PS1 mice were each divided in 2 groups, either receiving vehicle or SUL-138 via their food. Group size amounted 12 animals. Based on mouse weight of ˜30 g, food intake of ˜5 g/day and desired oral intake of 30 mg/day/kg, food pellets were sprayed with SUL-138 in water with 0.0145% ethanol at 1 g SUL-138 in 5 kg food. Vehicle food was prepared by spraying with the same volume of 0.0145% ethanol containing water.

Mice were treated chronically between 2.5 moa (pre-pathology/memory deficit) and 6 moa (age at which clear neuropathology and memory deficits occur) prior to testing.

FC: mice were exposed to a context for 2 min after which they received a 0.7 mA footshock. 30 sec after the footshock mice were out back in home cage. 24 h later mice were put in the same context and freezing levels were measured for 2 min.

LTP: Acute coronal hippocampal slices were kept in artificial CSF and LTP was measured after 3×100 Hz stimulation.

Phenotypers (provided by Sylics, Amsterdam, Netherlands): Mice were housed in the phenotypers for 3 days during which spontaneous behavior: activity, dark/light, habituation, kinematics, light dark phase transition pattern and sheltering were measured.

### Results

Overall welfare was monitored and did not show differences between vehicle and SUL-138 treated animals, with all groups showing similar increase in body weight.

Memory was assessed at 6 moa by measuring freezing following context acquisition.

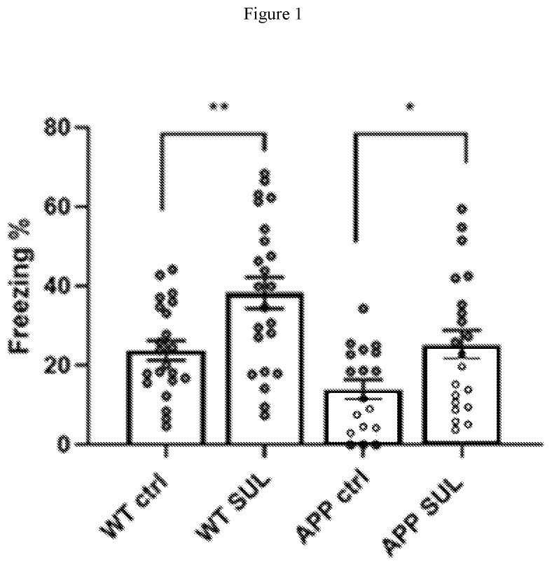

FIG. 1 shows how chronic SUL-138 treatment increases memory (Freezing %) in WT and APP mice. SUL-138 treatment increased freezing levels (memory) in both the WT and APP mice. Student's t-test, *: p<0.05 **: p<0.01.

APP/PS1 mice showed decreased freezing compared to WT mice when treated with control food, as expected. Upon chronic SUL-138 treatment memory in APP/PS1 mice was restored to WT levels. This shows that SUL-138 is effective in preventing or ameliorating Alzheimer's disease and/or its symptoms.

Interestingly, WT mice that received SUL-138 also performed better in the memory task. This indicates that SUL-138 is also effective for improving the memory function in a healthy mammal.

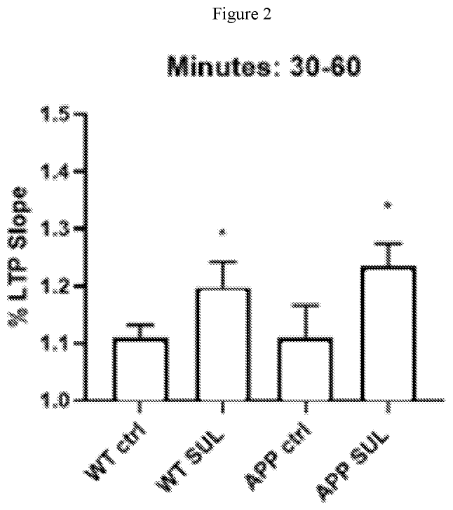

FIG. 2 shows how SUL-138 increases LTP maintenance in both WT and APP mice. Between 8-14 hippocampal slices per group (2A: WT ctrl, WT SUL-138, 2B: APP ctrl, APP SUL-138) received LTP evoked by 3×100 Hz stimulation (tetanus) of 1 sec separated by 20 sec. The slope was measured for 60 min. LTP was expressed as a percentage of baseline. All LTP data analysis was performed blinded. LTP maintenance (min 30-60) was significantly (p<0.05) higher in SUL-138 animals (both WT and APP); Student's t-test,* P<0.05; 2 C.

Chronic SUL-138 treatment did not induce differences in spontaneous behavior: activity, dark/light, habituation, kinematics, light dark phase transition pattern and sheltering were measured.

### Conclusions

The examples show SUL-138 to increase memory and LTP in both WT and APP/PS1 mice, and to effectively restore in APP/PS1 mice memory and LTP to control levels.

Increase in both these parameters reflects a general plasticity increasing/LTP facilitating process which is stimulated using SUL-138. This finding implies that SUL-138 may be used to relieve symptoms in neurological diseases that display reduced synaptic strength or plasticity.

SUL-138 effects seem specific for memory improvement, as treatment did not introduce atypical behavior in mice after chronic treatment for 3 m. In addition, no differences in weight were measured during 3 m of chronic oral treatment, that could indicate aversive or addictive behavior towards SUL-138-treated food, or changes in major physiological functions.

Finally, no animal welfare problems or differences between groups took place during the total experiment.

### Example 2

### Reduced Plaque Load in APP/PS1 Mice after Intervention by SUL-138

APP/PS1 (n=10) and wildtype mice (WT, n=10) mice were treated, either with vehicle or SUL-138. Mice were treated for 3 months starting at 3 months of age with either SUL-138 or vehicle treated food pellets. The mice were sacrificed at 6 months of age, the age at which (among others) hippocampal dependent memory impairment and apparent plaque load are expected.

4% PFA perfused brains, stored on sucrose, were sliced at 35 μM using a cryostat (−20° C.; Leica). Hippocampal slices (n=2/animal; 5 animals/group) were washed 3×10 min with 1× PBS, and then blocked for 1 h in Blocking solution (10 mL 1× PBS+500 μL normal Goat Serum+0.250 g Bovine Serum Albumin+20 μL Triton-100). The slices were incubated overnight with anti-Amyloid beta (6E10) (ITK Diagnostics, 1:400), washed 3×10 min with 1× PBS and then incubated with secondary Goat anti-mouse Alexa fluorescent 488 antibody (Sigma-Aldrich, 1:250) for 2 h. Then slices were washed 3×10 min with 1× PBS and mounted on slides.

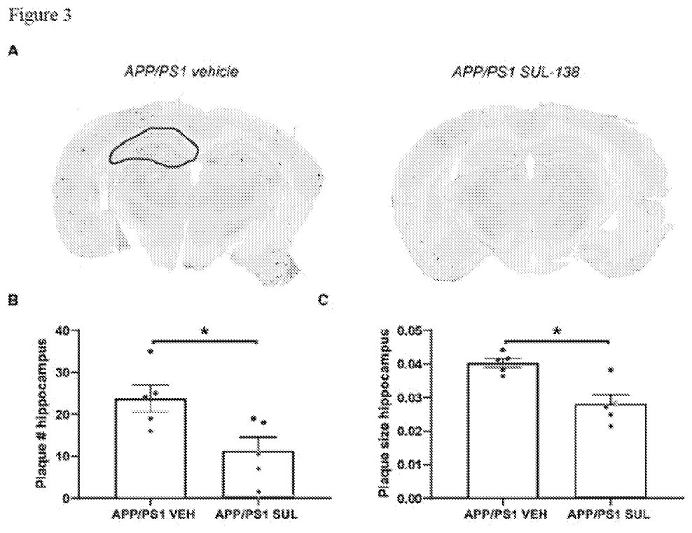

Slices were imaged using the Zeiss Cell Discover 7 high content microscope with LSM900 confocal head. Using Fiji, both hippocampi were selected separately for 5 animals per group (yellow line in FIG. 3A) and number and size of plaques were measured (FIG. 3B, C). The mean number of plaques and plaque size per animal were used for statistical analyses in GraphPad 8 using Student's t-test, one-sided.

Three months of oral SUL-138 reduced both the number of plaques (FIG. 3B; p=0.0138) and plaque size (FIG. 3C; p=0.0021) in APP/PS1 mice compared to vehicle treated mice. SUL-138 and vehicle treated WT animals did not show any plaques.

These data, together with SUL-138 rescuing memory and increasing synaptic transmission (long-term potentiation) in APP/PS1 mice observed according to example 1, show that SUL-138 is a potential therapeutic option against Alzheimer disease.

The bioavailability of SUL-138 brain appears to be high, thereby overcoming problems of other mitochondrial targeted compounds, making it a more suitable treatment option for future clinical application.

### Example 3

### In Vitro Assays Showing that Compounds According the Present Invention are Active

Excitotoxicity is the process wherein nerve cells suffer damage or death when the levels of otherwise necessary and safe neurotransmitters become pathologically high, resulting in the excessive stimulation of their receptors. Excitotoxicity may be involved in neurodegenerative diseases of the central nervous system such as Alzheimer's disease.

In vitro assays to investigate excitotoxicity utilize well-characterized inducers of neuronal cell death (e.g. glutamate, dopamine or NDMA) and the quantification of cell viability of stimulated neuronal-like cells. The human neuroblastoma-derived SH-SY5Y cell line can be differentiated in vitro to resemble mature neurons morphologically and biochemically. Moreover, the differentiated SH-SY5Y neuron-like cells are sensitive to excitotoxicity induced by, amongst others, glutamate and dopamine.

In this current study, the efficacy of SUL-11, SUL-127, SUL-13, SUL-138 (and its primary metabolite SUL-138M2), SUL-150 and SUL-151 to inhibit glutamate- and dopamine-induced excitotoxicity of human SH-SY5Y neuronal-like cells were investigated. SUL-11 is Trolox, while SUL-127 is the methyl ester of Trolox. These two compounds were used as reference.

The compounds used in this study are shown in Table 1, below:

TABLE 1

| | | | | |
|---|---|---|---|---|
| Compound | Chemical Name | Formula | Structure | MW |

Reference compounds

| | | | | |
|---|---|---|---|---|
| SUL-11 | 6-hydroxy-2,5,7,8- tetramethylchroman-2- carboxylic acid | C₁₄H₁₈O₄ | 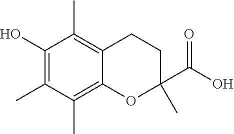 [SMILES: Cc1c(C)c2c(c(C)c1O)CCC(C)(C(=O)O)O2] | 250.3 |
| SUL-127 | methyl 6-hydroxy- 2,5,7,8-tetramethyl-3,4- dihydro-2H-1- benzopyran-2- carboxylate | C₁₅H₂₀O₄ | 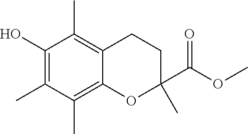 | 264.3 |

compounds according the invention

| | | | | |
|---|---|---|---|---|
| SUL-13 | (S)-6-hydroxy-2,5,7,8- tetramethyl-N-((R)- piperidin-3- yl)chromane-2- carboxamide | C₁₉H₂₈N₂O₃ | 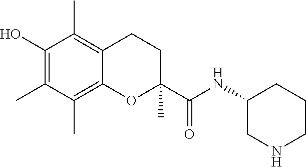 [SMILES: Cc1c(C)c2c(c(C)c1O)CC[C@@](C)(C(=O)N[C@@H]1CCCNC1)O2] | 332.4 |
| SUL-138 | (S)-(6-hydroxy-2,5,7,8- tetramethylchroman-2- yl)(4-(2-hydroxyethyl) piperazin-1- yl)methanone | C₂₀H₃₀N₂O₄ | 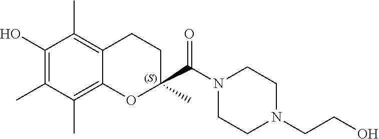 | 362.5 |
| SUL-138M2 | 4-(2,5-dihydroxy-3,4,6- trimethylphenyl)-2- hydroxy-1-(4-(2- hydroxyethyl)piperazin- 1-yl)-2-methylbutan-1- one | C₂₀H₃₂N₂O₅ | 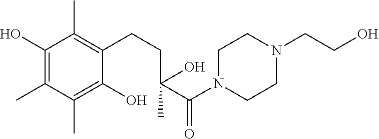 [SMILES: Cc1c(C)c(O)c(CC[C@@](C)(O)C(=O)N2CCN(CCO)CC2)c(C)c1O] | 380.5 |
| SUL-150 | (R)-(6-hydroxy-2,5,7,8- tetramethylchroman-2- yl)(piperazin-1- yl)methanone | C₁₈H₂₆N₂O₃ | 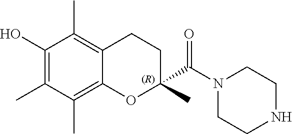 | 318.4 |
| SUL-151 | (S)-(6-hydroxy-2,5,7,8- tetramethylchroman-2- yl)(piperazin-1- yl)methanone | C₁₈H₂₆N₂O₃ | 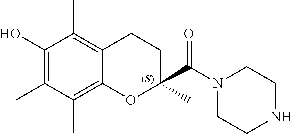 | 318.4 |

Human SH-SY5Y neuroblastoma cells (ATCC #CRL-2266) were maintained in DMEM medium containing 10% fetal bovine serum and 1% Penicillin-Streptomycin solution (#P4333, Sigma-Aldrich, St. Louis, Mo.) and passaged when the cultures reach a confluency of 70%. Prior to experiments, SH-SY5Y cells were differentiated by serum reduction (to 1%) and stimulation with 10 μM retinoic acid (#R7882, Sigma-Aldrich, St. Louis, Mo.) for 72 hours. Differentiated SH-SY5Y cells were seeded at 0.6·10⁵ cells/cm² for all experiments.

Differentiated SH-SY5Y cells were pre-incubated with SUL compounds (dose range 8·10⁻⁴ to 1·10⁻⁸ M) under standard culture conditions for 30 min and then stimulated with either 1-glutamate (60 mM; #12843-0, Sigma-Aldrich, St. Louis, Mo.) or dopamine (100 μM; #H8502, Sigma, St. Louis, Mo.) for an additional 24 h. Neutral Red Assay Solution (#N2889, Sigma-Aldrich, St. Louis, Mo.) was added to the cultures at a 10% (v/v) concentration during the final 4 hours of culture. Cells were washed with warm PBS and Neutral red solubilized in acid ethanol (1% acetic acid in 50% EtOH). Absorbances were recorded at 540 nm in a CLARIOStar Plus plate reader (BMG Labtech, Germany). Cell viability was normalized to absorbance measurements of untreated cultures (100% viable) and to absorbance measurements of samples that did not contain cells (0% viable).

All experiments were performed in triplicate per condition and averaged. Data obtained from two individual experiments were used for evaluation in GraphPad Prism 8.0 (GraphPad Software Inc, Ca). 4 parameter non-linear regression was used to determine the efficacy and potency of SUL compounds to reduce the excitotoxicity induced by either 1-glutamate or dopamine. The efficacy of SUL compounds to inhibit excitotoxicity was calculated as Eₘₐₓ=100*V_{(treated)}−V_{(vehicle)}/100%−V_{(vehicle)}), wherein V is the observed viability and Eₘₐₓ is the maximal effect evoked by SUL compound treatment.

No cellular toxicity was observed as a decrease in viability when using the SUL-compounds in the molar range shown in the table below.

SH-SY5Y neuroblastoma cells were differentiated into neuronal-like cells according to established protocols and stimulated with 60 mM glutamate to induce excitotoxicity. Glutamate decreased SH-SY5Y cell viability from 100±1.63% in vehicle-treated control cells to 55.4±1.7% in SH-SY5Y cells exposed to glutamate for 24 hours (p<0.0001). Pre-incubation of differentiated SH-SY5Y cells with SUL compounds (10⁻³ to 10⁻⁸M) dose-dependently increased cell viability of glutamate-challenged SH-SY5Y cells, albeit at different levels. Trolox and the methyl-ester of Trolox were clearly less effective than the other SUL-compounds, as shown in table 2 below.

Differentiated SH-SY5Y neuroblastoma cells were stimulated with 150 μM dopamine to induce excitotoxicity. Dopamine decreased SH-SY5Y cell viability from 100±0.8% in vehicle-treated control cells to 50.5±1.0% in SH-SY5Y cells exposed to dopamine for 24 hours (p<0.0001). Pre-incubation of differentiated SH-SY5Y cells with SUL compounds (10⁻³ to 10⁻⁸M) dose-dependently increased cell viability of dopamine-challenged SH-SY5Y cells, albeit at different efficacies, as shown in table 2 below. In this model, all compounds had decreased cell viability at the dose level of 10⁻³ M.

TABLE 2

| | Glutamate excitotoxicity | | Dopamine excitotoxicity | |
|---|---|---|---|---|
| Compound | EC₅₀ (M) | Emax (%) | EC₅₀ (M) | Emax (%) |
| SUL-11 | 4.62 · 10⁻⁶ | 76.7 | 8.35 · 10⁻⁶ | 79.5 |
| SUL-127 | 2.01 · 10⁻⁵ | 90.4 | 3.53 · 10⁻⁶ | 69.5 |
| SUL-13 | 3.82 · 10⁻⁶ | 100.0 | 3.24 · 10⁻⁷ | 91.9 |
| SUL-138 | 1.42 · 10⁻⁶ | 100.0 | 6.60 · 10⁻⁷ | 100.0 |
| SUL-138M2 | 4.43 · 10⁻⁶ | 100.0 | 1.69 · 10⁻⁶ | 94.7 |
| SUL-150 | 1.22 · 10⁻⁷ | 100.0 | 5.55 · 10⁻⁸ | 100.0 |
| SUL-151 | 9.61 · 10⁻⁸ | 100.0 | 6.92 · 10⁻⁸ | 100.0 |

The results in the table show that the SUL compounds according to the present invention exhibit either an improved EC50 (i.e. active at lower concentration), and/or improved Emax (i.e. the restoration of the toxicity is achieved at a higher level). Thereby, this example shown that next to SUL-138, also other SUL-compounds as claimed are likely to show the advantages of improved memory function and/or reduced plaque formation; i.e. in general are favorable in treating Alzheimer disease.

### Reference Experiment A

Hippocampal tissue from wildtype and APP/PS1 mice was examined for the protein expression of prostaglandin synthases and the thromboxane synthase A. Peptides resembling the prostaglandin synthase PTGS1, PTGES2, PTGES3, and PTGFS were found in the hippocampal tissue of both wildtype and APP/PS1 mice (Table 4). No protein fragments of PTGS2, PTGDS, PTGES1, PTGIS and TXA could be found. SUL-138 treatment of either wildtype or APP/PS1 mice did not alter the protein expression of prostaglandin synthesizing enzymes.

1. Compound according to formula (I) or (II), or a pharmaceutically acceptable salt thereof for use in the treatment of Alzheimer's disease, for improving the memory function, and/or for reducing plaque load in an Alzheimer disease patient; 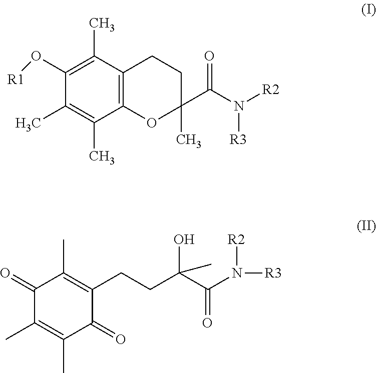
wherein R1 represents a hydrogen or prodrug moiety that can be removed in living tissue
and wherein either
R2 and R3 together with the N atom to which they are attached form a saturated or unsaturated, non-aromatic, optionally substituted 5-8 membered ring, having one to four N, O, or S atoms, wherein R2 and R3 together contain 3-12 carbon atoms;
or R2 is a hydrogen atom, or an alkyl group with 1-6 carbon atoms, and R3 is an alkyl group, optionally substituted with nitrogen or oxygen, wherein the alkyl group comprises 3-12 carbon atoms, the alkyl group in R3 comprises one or more non-aromatic cyclic structures and may contain linear and/or branched groups, and one or more ethylenic unsaturations.

2. Compound for use according to claim 1, wherein R1 is hydrogen or forms together with the 6-oxygen an ester group with 2-6 carbon atoms.

3. Compound for use according to any one of claims 1-2, wherein the nitrogen can be amine, quaternary amine, guanidine, or imine and oxygen is hydroxyl, carbonyl or carboxylic acid; and/or oxygen and nitrogen together form amide, urea or carbamate groups.

4. Compound for use according to any one of claims 1-3, wherein in either compounds according to formula (I) or according to formula (II), R2 and R3 together with the N atom to which they are attached form a saturated ring incorporating an additional N atom, which ring is unsubstituted or substituted with an alcohol, or alkanol group having 1-4 carbon atoms.

5. Compound for use according to claim 4, wherein the compound is a compound according to formula I.

6. Compound for use according to claim 5, wherein R2 and R3 together with the N atom to which they are attached form a 5-7 membered ring comprising one additional amine group, which ring is optionally substituted with methyl, ethyl, or alcohol substituted methyl or ethyl.

7. Compound for use according to any one of claims 1-3, wherein R2 is a hydrogen atom and R3 comprises a saturated cyclic structure having 4-7 carbon atoms and having one nitrogen atom, which ring may be substituted with an alkyl group, alcohol group, or with a group with 1-4 carbon atoms that may comprise an oxygen, carboxylic acid or amine group.

8. Compound for use according to claim 7, wherein the compound is a compound according to formula II and wherein R2 is a hydrogen atom and R3 comprises a cyclic structure having 4-6 carbon atoms and having one nitrogen atom which ring is optionally substituted with methyl, ethyl, or alcohol substituted methyl or ethyl.

9. Compound for use according to claim 1, wherein the compound is (6-hydroxy-2,5,7,8-tetramethylchroman-2yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13) or (6-hydroxy-2,5,7,8-tetramethylchroman-2-yl)(4-(2-hydroxyethyl)piperazin-1-yl)methanone (SUL-109).

10. Compound for use according to claim 9, wherein the compound is the S-enantiomer of SUL-109: S-(6-hydroxy-2,5,7,8-tetramethylchroman-2-yl)(4-(2-hydroxyethyl)piperazin-1-yl)methanone (SUL-138).

11. Compound for use according to any of claims 1-8, wherein the compound according formula (I) or formula (II) has a molecular weight lower than 500 Da.

12. Compound for use according to any of the preceding claims, wherein the use is for treating Alzheimer's disease.

13. Compound for use according to any of claims 1-12, wherein the use is for improving memory function.

14. Compound for use according to any of claims 1-13, wherein the use is for reducing plaque load in an Alzheimer disease patient.

15. Use of a compound as described in any one of claims 1-11 for improving the memory function in a mammal, preferably a human.
